(19) United States
(12) Patent Application Publication
(10) Pub. No.: US 2025/0011884 A1
(43) Pub. Date: Jan. 9, 2025

(54) Methods and Compositions for Identification and Modulation of Microbiome and Uses Thereof

(71) Applicants: University of Connecticut, Farmington, CT (US); Washington University, St. Louis, MO (US)

(72) Inventors: Yanjiao Zhou, Farmington, CT (US); Hanshu Yuan, New Britain, CT (US); Avraham Beigelman, Raanana (IL); Leonard Bacharier, Nashville, TN (US)

(73) Assignees: University of Connecticut, Farmington, CT (US); Washington University, St. Louis, MO (US)

(21) Appl. No.: 18/735,047

(22) Filed: Jun. 5, 2024

Related U.S. Application Data
(60) Provisional application No. 63/471,135, filed on Jun. 5, 2023.

(51) Int. Cl. C12Q 1/6895

(52) U.S. Cl. CPC C12Q 1/6895; C12Q 2600/118; C12Q 2600/156

(57) ABSTRACT

Disclosed herein are methods for prognosing the risk of progression of a disease or disorder associated with inflammation, e.g., asthma, in a subject. Such methods include detecting levels of a fungal species comprising Malassezia globosa in the subject.

### RELATED APPLICATION

This application claims the benefit of priority to U.S. Provisional Application No. 63/471,135, filed on Jun. 5, 2023, the entire contents of which are incorporated herein by reference.

### GOVERNMENT SUPPORT

This invention was made with government support under HL098115 awarded by the National Institutes of Health. The government has certain rights in the invention.

### SEQUENCE LISTING

The instant application contains a Sequence Listing which has been submitted electronically in XML file format and is hereby incorporated by reference in its entirety. Said XML copy, created on Aug. 23, 2024, is named 98121_00383_SL.xml and is 2,844 bytes in size.

### FIELD OF THE DISCLOSURE

The subject disclosure relates to methods, compositions, and systems for identification and modulation of the microbiome for the treatment or prevention of a disease or disorder wherein the disease or disorder is a disease or disorder associated with inflammation (for example, asthma or allergic diseases) that are effective and have less side effect. The subject disclosure particularly relates to the methods to establish relationship between the upper airway mycobiota and subsequent loss of control and exacerbation of respiratory disease such as asthma.

### BACKGROUND

Asthma is the most common chronic disease in childhood and is characterized by recurrent episodes of cough, wheezing, and shortness of breath. Early signs of loss of asthma control, often termed “yellow zone” (YZ), represents a period when the patient is at risk of progressing to severe exacerbation that requires oral corticosteroids (OCS) treatment. Prevention of asthma exacerbation is important, as it is strongly associated with asthma mortality and potentially inhibits lung growth. Factors related to YZ development and to progression to acute exacerbation in asthmatic children are not fully understood. Severe exacerbations may be life threatening and are associated with progressive loss of lung function. Asthma exacerbations have high impact on children, their families, the health care system, and may lead to subsequent decline in lung function. As asthma is associated with significant morbidity and mortality, there is a need to expand therapeutic options.

### SUMMARY

In one aspect, the present invention provides a method for prognosing the risk for progression of a disease or disorder associated with inflammation in a subject, comprising: (a) detecting the level of a fungal species in a biological sample from the subject, wherein the fungal species comprises Malassezia globosa, and (b) comparing the level of the fungal species in the biological sample with a predetermined threshold value; wherein the level of the fungal species above or below the predetermined threshold value indicates that the subject is at risk for progression of the disease or disorder associated with inflammation.

In some embodiments, the disease or disorder associated with inflammation is selected from the group consisting of asthma, inflammatory bowel disease, rheumatoid arthritis, juvenile rheumatoid arthritis, psoriatic arthritis, osteoarthritis, refractory rheumatoid arthritis, chronic non-rheumatoid arthritis, osteoporosis or bone resorption, coronary heart disease, atherosclerosis, endothelial dysfunction, vasculitis, ulcerative colitis, psoriasis, adult respiratory distress syndrome, diabetes, delayed-type hypersensitivity in skin disorders, endothelial dysfunction, or a combination thereof.

In some embodiments, the disease or disorder associated with inflammation is asthma.

In some embodiments, a decrease in the level of Malassezia globosa is indicative of progression of the disease or disorder associated with inflammation in the subject.

In some embodiments, the level of Malassezia globosa is detected by quantitative PCR and/or internal transcribed spacer (ITS) sequencing. In some embodiments, the level of Malassezia globosa is detected by ITS1 sequencing.

In some embodiments, the biological sample comprises a nasal saline wash, a nasal blow sample, a nasal mucus, a nasal discharge, a sinus mucus, or a sinus brushing comprising mucus from the surface of a sinus, or a bodily fluid.

In some embodiments, the method further comprises selecting a treatment regimen based on the risk of progression of the disease or disorder associated with inflammation in the subject.

In some embodiments, the treatment regimen is selected from the group consisting of oral corticosteroids (OCS), inhaled corticosteroids, leukotriene modifiers, anticholinergic agents, short-acting beta agonists, long-acting beta agonists, short-acting muscarinic antagonists, long-acting muscarinic antagonists, Theophylline, or any combination thereof.

In some embodiments, the method further comprises obtaining a biological sample from the subject.

In some embodiments, the subject is a human subject. In some embodiments, the subject has been previously diagnosed with the disease or disorder associated with inflammation. In some embodiments, the subject is concurrently diagnosed with the disease or disorder associated with inflammation. In some embodiments, the subject has been previously treated for the disease or disorder associated with inflammation. In some embodiments, the subject has not yet been treated for the disease or disorder associated with inflammation.

In another aspect, the present invention provides a method for prognosing the risk for progression of asthma in a subject, comprising: (a) detecting the level of a fungal species in a biological sample from the subject, wherein the fungal species comprises Malassezia globosa, and (b) comparing the level of the fungal species in the biological sample with a predetermined threshold value; wherein the level of the fungal species above or below the predetermined threshold value indicates that the subject is at risk for progression of asthma.

In some embodiments, the progression of asthma comprises an increased rate of annualized episodes of yellow zone (YZ) or a loss of asthma control.

In some embodiments, the progression of asthma comprises an increased risk of progression from YZ to severe asthma exacerbation.

In some embodiments, a decrease in the level of Malassezia globosa is indicative of progression of asthma in the subject.

In some embodiments, the level of Malassezia globosa is detected by quantitative PCR and/or internal transcribed spacer (ITS) sequencing. In some embodiments, the level of Malassezia globosa is detected by ITS1 sequencing.

In some embodiments, the biological sample comprises a nasal saline wash, a nasal blow sample, a nasal mucus, a nasal discharge, a sinus mucus, or a sinus brushing comprising mucus from the surface of a sinus, or a bodily fluid.

In some embodiments, the method further comprises a treatment regimen based on the risk of progression of asthma in the subject.

In some embodiments, the treatment regimen is selected from the group consisting of oral corticosteroids (OCS), inhaled corticosteroids, leukotriene modifiers, anticholinergic agents, short-acting beta agonists, long-acting beta agonists, short-acting muscarinic antagonists, long-acting muscarinic antagonists, Theophylline, or any combination thereof.

In some embodiments, the method further comprises obtaining a biological sample from the subject.

In some embodiments, the subject is a human subject. In some embodiments, the subject has been previously diagnosed with asthma. In some embodiments, the subject is concurrently diagnosed with asthma. In some embodiments, the subject has been previously treated for asthma. In some embodiments, the subject has not yet been treated for asthma.

In one aspect, the present invention provides a method for monitoring a disease or disorder associated with inflammation in a subject, comprising, (a) detecting the level of a fungal species in a first biological sample obtained at a first time from the subject having the disease or disorder associated with inflammation, wherein the fungal species comprises Malassezia globosa, and (b) detecting the level of the fungal species in a second biological sample obtained from the subject at a second time, wherein the second time is later than the first time; and (c) comparing the level of the fungal species in the second sample with the level of the fungal species in the first sample; wherein a change in the level of the fungal species is indicative of progression of the disease or disorder associated with inflammation in the subject.

In some embodiments, the disease or disorder associated with inflammation is selected from the group consisting of asthma, inflammatory bowel disease, rheumatoid arthritis, juvenile rheumatoid arthritis, psoriatic arthritis, osteoarthritis, refractory rheumatoid arthritis, chronic non-rheumatoid arthritis, osteoporosis or bone resorption, coronary heart disease, atherosclerosis, endothelial dysfunction, vasculitis, ulcerative colitis, psoriasis, adult respiratory distress syndrome, diabetes, delayed-type hypersensitivity in skin disorders, endothelial dysfunction, or a combination thereof.

In some embodiments, the disease or disorder associated with inflammation is asthma.

In some embodiments, a decrease in the level of Malassezia globosa is indicative of progression of the disease or disorder associated with inflammation in the subject.

In some embodiments, the level of Malassezia globosa is detected by quantitative PCR and/or internal transcribed spacer (ITS) sequencing. In some embodiments, the level of Malassezia globosa is detected by ITS1 sequencing.

In some embodiments, the first and the second biological samples comprise a nasal saline wash, a nasal blow sample, a nasal mucus, a nasal discharge, a sinus mucus, or a sinus brushing comprising mucus from the surface of a sinus, or a bodily fluid.

In another aspect, the present invention provides a method for treating or preventing a disease or disorder associated with inflammation in a subject, comprising administering to the subject an agent that increases the level of a fungal species, and wherein the fungal species comprises Malassezia globosa.

In one aspect, the present invention provides a method for treating or preventing a disease or disorder associated with inflammation in a subject, comprising increasing the level of a fungal species in the subject, and wherein the fungal species comprises Malassezia globosa.

In one aspect, the present invention provides a method for slowing down the progression of a disease or disorder associated with inflammation in a subject, comprising administering to the subject an agent that increases the level of a fungal species, and wherein the fungal species comprises Malassezia globosa.

In one aspect, the present invention provides a method for slowing down the progression of a disease or disorder associated with inflammation in a subject, comprising increasing the level of a fungal species in the subject, and wherein the fungal species comprises Malassezia globosa.

In some embodiments, the disease or disorder associated with inflammation is selected from the group consisting of asthma, inflammatory bowel disease, rheumatoid arthritis, juvenile rheumatoid arthritis, psoriatic arthritis, osteoarthritis, refractory rheumatoid arthritis, chronic non-rheumatoid arthritis, osteoporosis or bone resorption, coronary heart disease, atherosclerosis, endothelial dysfunction, vasculitis, ulcerative colitis, psoriasis, adult respiratory distress syndrome, diabetes, delayed-type hypersensitivity in skin disorders, endothelial dysfunction, or a combination thereof.

In some embodiments, the disease or disorder associated with inflammation is asthma.

In one aspect, the present invention provides a method for prognosing the risk for progression of a disease or disorder associated with inflammation in a subject, comprising: (a) detecting the level of a fungal species in a biological sample from the subject, wherein the fungal species comprises Malassezia globosa, (b) comparing the level of the fungal species in the biological sample with a predetermined threshold value; wherein the level of the fungal species below the predetermined threshold value indicates that the subject is at risk for progression of a disease or disorder associated with inflammation, and (c) selecting a treatment regimen based on the risk of progression of the disease or disorder associated with inflammation in the subject.

In one aspect, the present invention provides a method for prognosing the risk for progression of asthma in a subject, comprising: (a) detecting the level of a fungal species in a biological sample from the subject, wherein the fungal species comprises Malassezia globosa, (b) comparing the level of the fungal species in the biological sample with a predetermined threshold value; wherein the level of the fungal species below the predetermined threshold value indicates that the subject is at risk for progression of asthma, and (c) selecting a treatment regimen based on the risk of progression of asthma in the subject.

In one aspect, the present invention provides a kit for prognosing the risk of progression of a disease or disorder associated with inflammation in a subject, comprising one or more reagents for measuring the level of a fungal species in a biological sample from the subject, wherein the fungal species comprises Malassezia globosa, and a set of instructions for measuring the level of the fungal species.

In one aspect, the present invention provides a kit for prognosing the risk of progression of asthma in a subject, comprising one or more reagents for measuring the level of a fungal species in a biological sample from the subject, wherein the fungal species comprises Malassezia globosa, and a set of instructions for measuring the level of the fungal species.

These and other aspects of the present invention are described in more detail below.

### BRIEF DESCRIPTION OF THE DRAWINGS

The accompanying drawings are included to provide a further understanding of the methods and compositions of the disclosure, and are incorporated in and constitute a part of this specification. The drawings illustrate one or more embodiment(s) of the disclosure, and together with the description serve to explain the principles and operation of the disclosure.

FIGS. 1A-1B depict the upper airway fungal composition, distribution, and association with clinical variables at baseline. FIG. 1A is a pie chart showing the relative abundance of upper-airway fungal genera in nasal blow samples at baseline (n=194). FIG. 1B is a heatmap generated to identify upper-airway fungal pattern at baseline by hierarchical clustering and complete linkage approach. Fungal genera with relative abundance >0.1% are shown on the x-axis, and those with relative abundance <0.1% are assigned as “Others.”

FIGS. 2A-2D depict the fungal Malassezia species identified from nasal blow samples at baseline and associations between Malassezia species and clinical variables. FIG. 2A depicts the composition and abundance of Malassezia species in asthmatic children at baseline (n=194) as demonstrated by hierarchical clustering and complete linkage approach. FIG. 2B depicts the Pearson correlation between relative abundance of Malassezia globosa and age of enrollment. FIG. 2C depicts the Pearson correlation between relative abundance of Malassezia restricta and BMI. FIG. 2D depicts the relative abundance of two Malassezia restricta compared across the following races: African American, White, and more than 1 race. Statistical significances of mycobiome richness/diversity among clusters are tested using Kruskal Wallis rank-based nonparametric test. The Asian and Hispanic/Latino children were excluded in this analysis due to a limited number of samples (<2 for both). Each dot represents one participant. The horizontal line in the middle of the boxplot represents the median value of relative abundance of the mycobiome; the top and bottom horizontal line of boxplot represent the 25 and 75 percentiles of the data value.

FIGS. 3A-3C depict the associations between the relative abundance of Malassezia globosa at baseline/YZ and subsequent development of YZ or future asthma exacerbation. FIG. 3A depicts that the relative abundance of Malassezia globosa is correlated with annualized rate of YZ episodes. FIG. 3B depicts that higher relative abundance of Malassezia globosa is associated with longer time to developing YZ. FIG. 3C depicts the comparison of relative abundance of Malassezia globosa at YZ between OCS groups. (Yes: participants who have severe asthma exacerbation requiring OCS, No: participants have no severe asthma). Statistical significances of mycobiome relative abundance between groups are performed by Kruskal Wallis test.

FIGS. 4A-4D depicts the dynamic changes of the upper airway mycobiota from baseline to YZ. FIG. 4A depicts the changes in relative abundance of fungal genera in each asthmatic child. The relative abundance of the top 25 fungi at baseline and YZ. FIG. 4B depicts the significantly different fungi from baseline to YZ by DEseq2 (padj<0.05). FIG. 4C depicts the total fungal load and FIG. 4D depicts the fungal richness difference between baseline and YZ. Statistical differences are tested by Wilcoxon rank-sum test.

FIGS. 5A-5C depicts the fungi-bacteria correlation network at baseline and YZ. Bacteria and fungi correlations at baseline (FIG. 5A) and YZ (FIG. 5B). Relationships between bacterial genera and relative abundance of Malassezia species and other major fungal genera are determined by Spearman correlation after central log ratio transformation of relative abundance of bacteria or fungi. *p<0.05; **p<0.01. Red and blue colors represent negative and positive correlations, respectively. FIG. 5C depicts the Pearson correlation between changes of fungal diversity and changes of bacterial diversity from baseline to YZ.

FIG. 6 depicts the scheme of sample collection. Green (first) rectangle represents the timepoints when children's asthma is well controlled (baseline), yellow (middle) rectangle represents when children experience first episode of loss of asthma control (YZ), and red (last) rectangle represents when children have asthma exacerbation requiring OCS treatment. The maximum follow-up time for children included in fungal analysis is up to 15 months.

FIG. 7A depicts the Pearson's Correlation between Shannon diversity and BMI (Body Mass Index). FIG. 7B depicts the Pearson's Correlation between Shannon diversity and age of enrollment (5-11 years of age). Both p values are less than 0.05 after FDR adjustment.

FIG. 7C depicts the total fungal load difference between children with or without pet exposure at baseline. Y-axis represents 18S rRNA million copies per microliter estimated by qPCR. Children exposed to pets tended to have higher fungal load (p=0.028), but this became non-significant after FDR adjustment (padj=0.168).

FIG. 8A depicts the correlation between log transformed absolute abundance of Malassezia globosa with annualized rate of YZ episodes. The results are controlled for age, pets, BMI, virus presence, and sex. FIG. 8B depicts the comparison of log transformed absolute abundance of Malassezia globosa at baseline between OCS groups. (Yes: participants who have severe asthma exacerbation requiring OCS, No: participants have no severe asthma). FIG. 8C depicts the comparison of relative abundance of Malassezia globosa at baseline between OCS groups. FIG. 8D depicts the comparison of log transformed absolute abundance of Malassezia globosa at YZ between OCS groups.

FIG. 9A depicts the correlation of the relative abundance of Malassezia restricta and the annualized rate of YZ episodes. Comparison of relative abundance of Malassezia restricta at baseline (FIG. 9B) and YZ (FIG. 9C) between OCS groups. Statistical significances of Malassezia restricta relative abundance between groups are performed by Kruskal Wallis test or Wilcoxon test.

FIG. 10 depicts no differences of relative abundance of Malassezia species in virus-positive and -negative samples at baseline and YZ.

### DETAILED DESCRIPTION

Before the disclosed processes and materials are described, it is to be understood that the aspects described herein are not limited to specific embodiments, or examples, and as such can, of course, vary. It is also to be understood that the terminology used herein is for the purpose of describing particular aspects only and, unless specifically defined herein, is not intended to be limiting.

Accumulating evidence suggests that the upper airway bacterial microbiota is implicated in asthma inception, severity, and exacerbation. Unlike bacterial microbiota, the role of the upper airway fungal microbiome (mycobiome) in asthma control is poorly understood. The upper airway commensal mycobiome is associated with future asthma control. This work highlights the importance of the mycobiota in asthma control and may contribute to the development of fungi-based markers to predict asthma exacerbation. It was found that two Malassezia species, Malassezia globosa and Malassezia restricta, were the major mycobiome components in the upper airways of asthmatic children. Relative abundance of Malassezia globosa at asymptomatic baseline was inversely correlated with future loss of asthma control. Higher relative abundance of Malassezia globosa at the time of loss of asthma control was inversely correlated with future severe asthma exacerbation. The upper airway mycobiome underwent significant changes from baseline to time of loss of asthma control. There was a complex correlation between fungal mycobiome and bacterial microbiome in the upper airways of asthmatic children.

Disclosed herein is a new method for preventing or treating a disease or disorder in a subject in need thereof, wherein said disease or disorder is a disease associated with inflammation (for example, asthma or allergic diseases), the method including: detecting or identifying levels of a fungal species comprising Malassezia globosa in the subject; predicting a future loss of control and exacerbation of the disease, for example, asthma control and asthma exacerbation; and adjusting a treatment for the subject accordingly. Advantageously, this method results in improved diagnosis and treatment regimens for respiratory diseases. This is the first disclosure of correlation of fungal species in upper airway Malassezia globosa with future loss of control and exacerbation of respiratory diseases such as asthma. Thus, the disclosed method results in improved diagnosis and treatment regimens for respiratory diseases. The methods and techniques disclosed may be applied to improve diagnosis and therapeutics for various respiratory diseases such as asthma.

The human upper airway is a unique ecological niche that is colonized by a collection of bacteria, viruses, and fungi that interact with each other and with the host. Along with upper airway mucosa, upper airway microorganisms serve as the first line of defense against respiratory pathogens. Accumulating evidence strongly indicate that upper airway microbiota are associated with asthma inception, severity, and exacerbations. Our recent work involving a large cohort of school-aged asthmatic children who are receiving low-dose inhaled corticosteroids (ICS), demonstrated that higher relative abundance of upper airway Staphylococcus or Moraxella during periods of well-controlled asthma was associated with an increased risk of subsequent loss of asthma control (episodes of YZ), while higher relative abundance of the commensal bacteria Corynebacterium and Dolosigranulum were associated with decreased risk of subsequent loss of asthma control. However, the fungal component of the human upper airway microbiome and its role in asthma control has not been studied.

The collection of fungal species within the body, designated as “mycobiota”, is an important component of the airway microbiome in humans. Our understanding of mycobiota in asthma is largely restricted to several pathogenic fungi that grow at human body temperature and potentially infect the lungs. Aspergillus is one type of airborne fungus that causes allergic bronchopulmonary aspergillosis (ABPA), which can contribute to poorly controlled asthma. Penicillium, Pneumocystis, and other indoor and outdoor fungal allergens also contribute to respiratory fungal infection and severe forms of allergic asthma. One study found that up to 30% of asthmatic children and up to 60% of children with severe asthma were sensitized to these environmental fungi. In addition, higher Mucor exposure in the homes of urban inner-city children were associated with difficult-to-control asthma. With the development of high throughput sequencing, a comprehensive characterization of the commensal mycobiome in asthma is feasible. With a relatively small number of samples, a recent study showed that upper airway fungi are associated with respiratory illness in asthmatic children. However, other than these few reports and despite the strong potential implications of fungi in respiratory infection and asthma severity, little is known about the characteristics of the commensal mycobiome in the upper airway in asthmatic children and their relationship with loss of asthma control and exacerbation.

This disclosure is related to determining the relationship between the upper airway mycobiota and subsequent loss of asthma control and exacerbation of asthma. Leveraging a large, well-characterized prospective clinical trial, the present inventors analyzed the mycobiota from 301 nasal blow samples collected from asthmatic children at baseline and at YZ, and reported, for the first time, the composition and dynamics of the upper airway mycobiota in asthmatic children. A specific fungal species, Malassezia globosa, was associated with future asthma activities.

In an aspect, disclosed is a method for preventing or treating a disease or disorder in a subject in need thereof, wherein said disease or disorder is a disease associated with inflammation (for example, asthma or allergic diseases), wherein the method includes detecting/identifying levels of a fungal species including Malassezia globosa in the subject; predicting a future loss of control and exacerbation of the disease, for example, asthma control and asthma exacerbation; and adjusting a treatment for the subject accordingly. In an aspect, a correlation of fungal species such as Malassezia globosa in upper airway of a subject is used to predict future loss of control and exacerbation of respiratory diseases such as asthma. The disclosed methods result in improved diagnosis and treatment regimens for respiratory diseases such as asthma.

In an aspect, disclosed is a method of detecting an upper airway bacterial microbiota in a subject who has a disease associated with inflammation such as asthma, the method including detecting fungal species, or a proportion of fungal species, in a biological sample from the subject that are in one of or any combination of the following fungi: Malassezia, Blumeria, Aureobasidium, Penicillium, Aspergillus, Candida, Cladosporium, and/or Alternaria. In an embodiment, the biological sample is a nasal saline wash, a bodily fluid, nasal mucus, nasal discharge, sinus mucus, or sinus brushing comprising mucus from the surface of a sinus.

“Biological sample” or “sample” refers to materials obtained from or derived from a subject or patient. In embodiments, a biological sample is or includes a bodily fluid such as nasal discharge or mucus. In embodiments, a biological sample is or includes a wash, such as a saline wash, e.g., a nasal saline wash. In embodiments, the biological sample is or includes mucus. In embodiments, the mucus is sinus mucus (e.g., mucus obtained or collected from the surface of a sinus). In embodiments, the biological sample is a sinus brushing including mucus from the surface of a sinus. In embodiments, a biological sample is or includes sputum, phlegm, saliva, or mucus. In embodiments, a biological sample is or includes blood, serum, or plasma. In embodiments, a biological sample is or includes blood, a blood fraction, or product (e.g., serum, plasma, platelets, red blood cells, and the like). In embodiments, a biological sample is or includes tissue, such as nasal or sinus tissue. In embodiments, a sample is obtained from a eukaryotic organism, such as a mammal such as a primate e.g., chimpanzee or human; cow; dog; cat; a rodent, e.g., guinea pig, rat, or mouse; rabbit; or a bird; reptile; or fish. In embodiments, a biological sample includes sections of tissues such as biopsy and autopsy samples, and frozen sections taken for histological purposes.

In an embodiment, the subject has nasal dysbiosis or sinus dysbiosis. As used herein the term “dysbiosis” means a difference in the microbiota compared to a general or healthy population. The term “nasal dysbiosis” means a difference in the nasal microbiota compared to a general or healthy population. In embodiments, dysbiosis includes a difference in nasal microbiota commensal species diversity compared to a general or healthy population. In embodiments, dysbiosis includes a decrease of beneficial microorganisms and/or increase of pathobionts (pathogenic or potentially pathogenic microorganisms) and/or decrease of overall microbiota species diversity. Many factors can harm the beneficial members of the nasal microbiota leading to dysbiosis, including (but not limited to) infection, antibiotic use, psychological and physical stress, radiation, and dietary changes. In embodiments, the dysbiosis includes a reduced amount (absolute number or proportion of the total microbial population) of bacterial or fungal cells of a species or genus (e.g., 5%, 10%, 15%, 20%, 25%, 30%, 35%, 40%, 45%, 50%, 55%, 60%, 65%, 70%, 75%, 80%, 85%, 90%, 95% or more lower) compared to a healthy subject (e.g., a corresponding subject who does not have asthma or an infection, and who has not been administered an antibiotic within about 1, 2, 3, 4, 5, or 6 months, and/or compared to a general or healthy population). In embodiments, the dysbiosis includes an increased amount (absolute number or proportion of the total microbial population) of bacterial or fungal cells within a species or genus (e.g., 5%, 10%, 15%, 20%, 25%, 30%, 35%, 40%, 45%, 50%, 55%, 60%, 65%, 70%, 75%, 80%, 85%, 90%, 95% or more higher) compared to a healthy subject (e.g., a corresponding subject who does not have asthma or an infection, and who has not been administered an antibiotic within about 1, 2, 3, 4, 5, or 6 months, and/or compared to a general or healthy population).

In an embodiment, the method included determining whether the subject has an increased proportion of nasal microbiota in the Malassezia genus of fungi compared to a general population of subjects who have asthma. In an embodiment, the method included determining whether the subject has an increased proportion of Malassezia globosa compared to a general population of subjects who have asthma. In an embodiment, the method included determining whether the subject has a decreased proportion of Malassezia globosa compared to a general population of subjects who have asthma.

In an aspect, disclosed is a method of treating or preventing a condition in a subject in need thereof, the method including detecting/identifying levels of a fungal species including Malassezia globosa in the subject; and administering to the subject an effective amount of an antibiotic compound, wherein: (a) the condition is asthma, an asthma exacerbation, or a rhinovirus infection; (b) the condition is asthma, an asthma exacerbation, or a rhinovirus infection, and the method comprises detecting nasal dysbiosis in the subject; (c) the condition is asthma, an asthma exacerbation, or a rhinovirus infection, and the subject has been identified as having an increased risk of asthma exacerbation or rhinovirus infection compared to a general population of subjects who have asthma; (d) the condition is chronic rhinosinusitis or nasal polyposis; (c) the condition is chronic rhinosinusitis or nasal polyposis, and the method comprises detecting sinus dysbiosis in the subject; (f) the condition is chronic rhinosinusitis or nasal polyposis, and the subject has been identified as having or being at risk of having chronic rhinosinusitis or nasal polyposis.

The present invention provides methods for prognosing the risk for progression of a disease or disorder associated with inflammation in a subject, comprising: (a) detecting the level of a fungal species in a biological sample from the subject, wherein the fungal species comprises Malassezia globosa, and (b) comparing the level of the fungal species in the biological sample with a predetermined threshold value; wherein the level of the fungal species above or below the predetermined threshold value indicates that the subject is at risk for progression of the disease or disorder associated with inflammation.

In another aspect, the present invention provides a method for prognosing the risk for progression of a disease or disorder associated with inflammation in a subject, the method comprising: (a) detecting the level of a fungal species in a biological sample from the subject, wherein the fungal species comprises Malassezia fungal species comprising Malassezia globosa, (b) comparing the level of the fungal species in the biological sample with a predetermined threshold value; wherein the level of the fungal species below the predetermined threshold value indicates that the subject is at risk for progression of the disease or disorder associated with inflammation, and (c) selecting a treatment regimen based on the risk of progression of the disease or disorder associated with inflammation in the subject.

In another aspect, the present invention provides methods for monitoring a disease or disorder associated with inflammation in a subject, comprising, (a) detecting the level of a fungal species in a first biological sample obtained at a first time from the subject having the disease or disorder associated with inflammation, wherein the fungal species comprises Malassezia globosa, and (b) detecting the level of the fungal species in a second biological sample obtained from the subject at a second time, wherein the second time is later than the first time; and (c) comparing the level of the fungal species in the second sample with the level of the fungal species in the first sample; wherein a change in the level of the fungal species is indicative of progression of the disease or disorder associated with inflammation in the subject.

In another aspect, the present invention provides a method for monitoring a disease or disorder associated with inflammation in a subject, the method comprising: (a) detecting the level of a fungal species in a first biological sample obtained at a first time from the subject, wherein the fungal species comprises Malassezia globosa, and (b) detecting the level of the fungal species in a second biological sample obtained from the subject at a second time, wherein the second time is later than the first time; (c) comparing the level of the fungal species in the second sample with the level of the fungal species in the first sample; wherein a decrease in the level of the fungal species is indicative of progression of the disease or disorder associated with inflammation in the subject, and (d) selecting a treatment regimen based on the risk of progression of the disease or disorder associated with inflammation in the subject.

In certain embodiments, the disease or disorder is inflammation in combination with the disease associated with inflammation, wherein the disease associated with inflammation is inflammatory bowel disease, rheumatoid arthritis, juvenile rheumatoid arthritis, psoriatic arthritis, osteoarthritis, refractory rheumatoid arthritis, chronic non-rheumatoid arthritis, osteoporosis or bone resorption, coronary heart disease, atherosclerosis, endothelial dysfunction, vasculitis, ulcerative colitis, psoriasis, adult respiratory distress syndrome, diabetes, delayed-type hypersensitivity in skin disorders, asthma, endothelial dysfunction, or a combination thereof. In certain embodiments, the disease associated with inflammation is asthma. In certain embodiments, the subject is human.

In another aspect, the present invention provides a method for prognosing the risk for progression of asthma in a subject, comprising: (a) detecting the level of a fungal species in a biological sample from the subject, wherein the fungal species comprises Malassezia globosa, and (b) comparing the level of the fungal species in the biological sample with a predetermined threshold value; wherein the level of the fungal species above or below the predetermined threshold value indicates that the subject is at risk for progression of asthma.

In another aspect, the present invention provides a method for prognosing the risk for progression of asthma in a subject, comprising: (a) detecting the level of a fungal species in a biological sample from the subject, wherein the fungal species comprises Malassezia fungal species comprising Malassezia globosa, (b) comparing the level of the fungal species in the biological sample with a predetermined threshold value; wherein the level of the fungal species below the predetermined threshold value indicates that the subject is at risk for progression of asthma, and (c) selecting a treatment regimen based on the risk of progression of asthma in the subject.

In some embodiments, the progression of asthma comprises an increased rate of annualized episodes of yellow zone (YZ) or a loss of asthma control. In some embodiments, the progression of asthma comprises an increased risk of progression from YZ to severe asthma exacerbation.

In some embodiments, the level of Malassezia globosa is detected or determined by quantitative PCR (qPCR), e.g., qPCR specifically targeting Malassezia globosa. In some embodiments, the level of Malassezia globosa is detected or determined by internal transcribed spacer (ITS) sequencing, e.g., ITS1 sequencing.

In certain embodiments of the methods provided herein, an increase or decrease in the level of the fungal species in the biological sample as compared to the level of the fungal species in a control sample is an indication that the subject is at risk for progression.

In certain embodiments of the methods provided herein, an increase, or decrease in the level of the fungal species in the second sample as compared to the level of the fungal species in the first sample is an indication that the subject is at risk for progression, it is also an indication for selection of more aggressive and/or preventive treatment in the subject. In certain embodiments of the methods provided herein, an increase or decrease in the detected level of the fungal species in the second sample as compared to the level of the fungal species in the first sample is an indication that the subject is not at risk for progression, it is also an indication against selection of more aggressive and/or preventive treatment in the subject.

In some embodiments, the fungal species is Malassezia globosa. In some embodiments, a decrease in the level of Malassezia globosa is indicative of progression of the disease or disorder associated with inflammation, e.g., asthma, in the subject.

In some embodiments, the methods further comprise obtaining a biological sample from the subject. The present invention may be practiced with any suitable biological sample. In some embodiments, the biological sample may be obtained from sources that include whole blood, serum, urine, diseased and/or healthy organ tissue. In certain embodiments, the biological sample comprises a nasal saline wash, a nasal blow sample, a nasal mucus, a nasal discharge, a sinus mucus, or a sinus brushing comprising mucus from the surface of a sinus, or a bodily fluid. Any commercial device or system for isolating and/or obtaining suitable biological samples, e.g., nasal blow samples, and/or for processing said materials prior to conducting a detection reaction is contemplated.

The methods further comprise selecting a treatment regimen based on the risk of progression of the disease or disorder associated with inflammation in the subject. In one embodiment, a treatment regimen known to be effective against the disease or disorder associated with inflammation, e.g., asthma, is selected for the subject. In certain embodiments, the treatment method is started, change, revised, or maintained based on the results from the prognostic or monitoring methods of the invention, e.g., when it is determined that the subject is at risk for disease progression, when it is determined that the subject is responding to the treatment regimen, or when it is determined that the subject is not responding to the treatment regimen, or when it is determined that the subject is insufficiently responding to the treatment regimen. In certain embodiments, the treatment method is changed based on the results from the prognostic or monitoring methods.

In some embodiments, the treatment regimen is selected from the group consisting of oral corticosteroids (OCS), inhaled corticosteroids, leukotriene modifiers, anticholinergic agents, short-acting beta agonists, long-acting beta agonists, short-acting muscarinic antagonists, long-acting muscarinic antagonists, and Theophylline; or any combination thereof.

In certain embodiments, the methods provided herein further comprise introducing one or more specific treatment regimens for the subject based on the results of the prognostic and monitoring methods provided herein.

In yet other embodiments, the methods provided herein further comprise the step of administering a therapeutically effective amount of a therapy based on the results of the prognostic and monitoring methods provided herein.

The present invention also provides methods for treating and/or preventing a disease or disorder associated with inflammation, e.g., asthma, in a subject, comprising administering to the subject an agent that increases the level of a fungal species, and wherein the fungal species comprises Malassezia globosa.

In another aspect, the present invention provides a method for treating or preventing a disease or disorder associated with inflammation, e.g., asthma, in a subject, comprising increasing the level of a fungal species in the subject, and wherein the fungal species comprises Malassezia globosa.

In one aspect, the present invention provides a method for slowing down the progression of a disease or disorder associated with inflammation, e.g., asthma, in a subject, comprising administering to the subject an agent that increases the level of a fungal species, and wherein the fungal species comprises Malassezia globosa.

In yet another aspect, the present invention provides a method for slowing down the progression of a disease or disorder associated with inflammation, e.g., asthma, in a subject, comprising increasing the level of a fungal species in the subject, and wherein the fungal species comprises Malassezia globosa.

In some embodiments, the subject is a human subject. The subject may have been previously diagnosed with the disease or disorder associated with inflammation, e.g., asthma, or is concurrently diagnosed with the disease or disorder associated with inflammation, e.g., asthma. In some embodiments, the subject has been previously treated for the disease or disorder associated with inflammation, e.g., asthma. In other embodiments, the subject has not yet been treated for the disease or disorder associated with inflammation, e.g., asthma.

The invention also provides compositions and kits for prognosing or monitoring a disease or disorder, progression or recurrence of a disease or disorder associated with inflammation, e.g., asthma. These kits may include one or more reagents for measuring the level of a fungal species in a biological sample from the subject, and a set of instructions for measuring the level of the fungal species.

The present disclosure is illustrated and further described in more detail with reference to the following non-limiting examples.

### Examples

### Materials and Methods

### Study Design

This microbiome study was coupled to the Step Up Yellow Zone Inhaled Corticosteroids to Prevent Exacerbations (STICS; NCT02066129) clinical trial, conducted by the NHLBI's AsthmaNet. The STICS clinical trial was approved by the AsthmaNet steering committee, protocol review committee, and data and safety monitoring board (DSMB). The trial was independently approved by each of the IRB of the clinical sites. Informed consent from all participants were obtained.

The STICS clinical trial recruited school age children with mild asthma who were being treated with daily low-dose ICS. Detailed of the STICS clinical trial study design, YZ criteria, clinical information of study participants, and the outcomes can be found in a previous publication². In brief, 254 children, 5-11 years of age and from different sites across the US, were treated for 48 weeks with low-dose inhaled glucocorticoids. YZ episodes were identified by the occurrence of any of the following: the use of two doses (four inhalations) of rescue albuterol in 6 hours, the use of three doses (six inhalations) of rescue albuterol in 24 hours, or one night awakening that was due to asthma that was treated with albuterol. When the children had early signs of loss of asthma control (YZ), children were randomly treated with either the same dose of ICS (1×ICS), or quintupled dose of ICS (5×ICS) for 7 days.

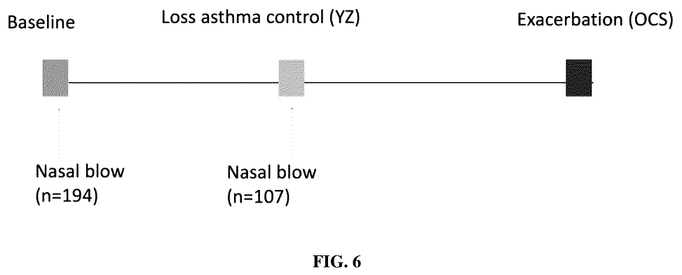

Nasal blow samples were collected in children at the randomization visit (baseline) when asthma symptoms were well-controlled, and at the time of the first episode of early signs of loss of asthma control (YZ) (FIG. 6). The samples from the YZ timepoint were collected before ICS treatment to avoid potential influence of high dose ICS to nasal microbiota. YZ samples collected by parents at home followed the sample collection protocol received at the randomization visit. Nasal samples were used for characterization of the bacterial microbiome previously, and fungal microbiome in the current study.

### ITS1 Sequencing and Data Processing

Total nucleic acid was extracted from nasal blow samples (200 μl) using the bioMerieux NucliSENS easyMAG automated extractor kit following standard protocol. Negative and positive controls were included in each sequence run. Internal transcribed spacer 1 (ITS1) region of fungal genomic DNA were amplified using primers: 18S-F-GTAAAAGTCGTAACAAGGTTTC (SEQ ID NO: 1) and 5.8S-IR-GTTCAAAGAYTCGATGATTCAC (SEQ ID NO: 2), followed by sequencing on an Illumina Miseq (2× 300 bp) platform. ITS1 was chosen, as it captures a more diverse fungal population than ITS2 and 18S rRNA. ITS1 sequencing will generate relative abundance of M. globosa.

Raw sequencing reads were demultiplexed using Illumina software. Reads in each sample were processed using DADA2 pipeline (V1.16) for taxonomy assignment to amplicon sequence variants (ASVs) based on UNITE database (Version 8.3). A classification with a confidence of <0.5 was assigned to “unclassified”. To additionally assign taxonomy of unclassified fungal genera, we aligned all ASVs that were unclassified at the genus level to Nucleotide (NT) database using BLAST+2.7.1. A given ASV was assigned to a specific genus based on NT taxonomy if the alignment of an ASV read was >95% identity and 95% coverage to the NT database.

Samples with less than 5000 reads were removed from the analysis. The ITS1 sequencing reads from the remaining samples were rarefied to 5000 reads/sample and transformed to relative abundance. Taxa with mean relative abundance of >0.1% were used for downstream analysis. Additionally, taxa identified as plant based on NCBI taxonomy were filtered out, including Solanum, Dioscorea, Pisum, Sinapis, Daucus, Cicer, Trifolium, Glycine, and Prunus. All the taxa found within negative controls (water and extraction controls) were less than 0.1% in nasal wash samples; therefore, our analysis results were not impacted by potential contamination from negative controls.

### Quantification of Fungal Load

FungiQuant assay was performed to quantify the fungal 18S rRNA gene copy. Based on the qPCR approach, a Candida albicans 18S rRNA gene clone was used as quantification standard. A qPCR specifically targeting M. globosa can be used to quantify the absolute abundance of M. globosa.

### Statistical Analysis of the Mycobiota

Hierarchical clustering using complete linkage was used to explore the mycobiota patterns among all participants. Fungal genera with relative abundance >0.1%, along with the remaining genera aggregated as ‘Others’ were included in the construct of clustering and generating a heatmap. Clustering and a heatmap of Malassezia species at baseline were also produced. Pearson's correlation analysis was used to examine associations between mycobiota characteristics (relative abundance, richness, Shannon diversity, fungal load) and continuous clinical variables. Kruskal-Wallis test or Wilcoxon signed-rank test were used to test associations between mycobiota characteristics with categorical clinical variables. Linear regression analysis was performed for modelling the relationship between the annualized episodes of YZ and with relative abundance of Malassezia species. In the model, annualized YZ episodes were treated as the response variable; the relative abundance of Malassezia species was the predictor; and age, gender, BMI, virus presence in nasal blow samples, or pet exposure were covariates.

Cox proportional-hazards analysis assessed the association between time for YZ development (response) and log-transformed relative abundance of the fungal genera (predictor). The association was examined by likelihood ratio test and was visualized via Kaplan-Meier curves. Because the predictor (relative abundance of Malassezia species) was a continuous variable and Kaplan-Meier only handle categorical predictor, Malassezia species were divided into the high abundance group and the low abundance group, with the best cut point that led to the largest difference in survival by log rank test (each group has at least 30% of samples). The proportional Hazards assumption was tested via scaled Schoenfeld residuals and was not rejected. The model stability was examined by removing two samples with slightly larger dfbeta, which led to consistent conclusions.

To test the robustness of the results on association between Malassezia species and future loss of asthma control or asthma exacerbation, and to ensure results are not driven by data from one geographical location/sample collection site, sensitivity tests were performed by excluding samples from one of nine geographic locations, so that each sensitivity test used samples from the 8 remaining geographic locations.

To identify specific fungi (>0.1% relative abundance) that statistically differ between baseline and YZ, differential analyses were performed based on the negative binomial distribution using the DESeq2 package (version 1.34.0). It was further inspected the results by plotting raw and relative abundance of the fungal data and removed any differential fungal taxa that were likely driven by one or two samples.

To determine correlations between bacteria and fungi at baseline or YZ, centered log ratio transformation of relative abundance of fungi or bacteria was first performed. Bacterial genera with mean relative abundance greater than 1% and fungal genera with mean relative abundance greater than 0.5% were selected for Spearman correlation. All bacteria-fungi correlations were plotted using corrplot package in R.

All above analyses were performed in R (R version 4.1.2). A p value of <0.05 was considered statistically significant. P values were adjusted by false discovery rate (FDR) when multiple comparisons were involved. These include association analysis between fungi and multiple clinical factors. An adjusted p value <0.1 was considered statistical significance.

### Data Availability

Raw sequencing of this ITS1 dataset is available from the SRA database with the accession number PRJNA830491.

### Results

### Mycobiota Composition in Upper Airway at Baseline

In total, 374 nasal blow samples from 214 asthmatic children were collected and subjected to ITS1 sequencing. After data processing, 301 samples (194 at baseline and 107 at YZ) yielded more than 5000 reads/sample and were used for downstream analyses. The demographics and clinical characteristics of these children were shown in Table 1.

TABLE 1
Characteristics of participants at baseline

| | Mean ± standard | |
|---|---|---|
| Demographics and clinical characteristics | deviation/Frequency | |
| (N = 214) | (Percentage) | |
| Age at enrollment - year | 8.1 ± 1.9 | |
| BMI | 18.5 ± 4.0 | |
| Male sex -no. (%) | 135 | (63.1%) |
| Hispanic ethnic group - no. (%) | 66 | (30.8%) |
| Race - no. (%) | | |
| Black | 49 | (22.3%) |
| White | 116 | (54.2%) |
| Other race | 49 | (22.3%) |
| No. of patients who develop yellow zone - no. (%) | 149 | (69.6%) |
| No. of patients who use oral corticosteroid - no. (%) | 26 | (26.1%) |
| Tobacco smoke exposure - no. (%) | 83 | (38.8%) |
| No. of patients who have pet exposure - no. (%) | 117 | (54.6%) |
| No. of patients who have virus presence | 69 | (32.2%) |
| No. of patients who have previous antibiotic use | 72 | (33.6%) |
| Season - no. (%) | | |
| Spring | 39 | (18.2%) |
| Summer | 46 | (21.5%) |
| Fall | 72 | (33.6%) |
| Winter | 57 | (26.6%) |

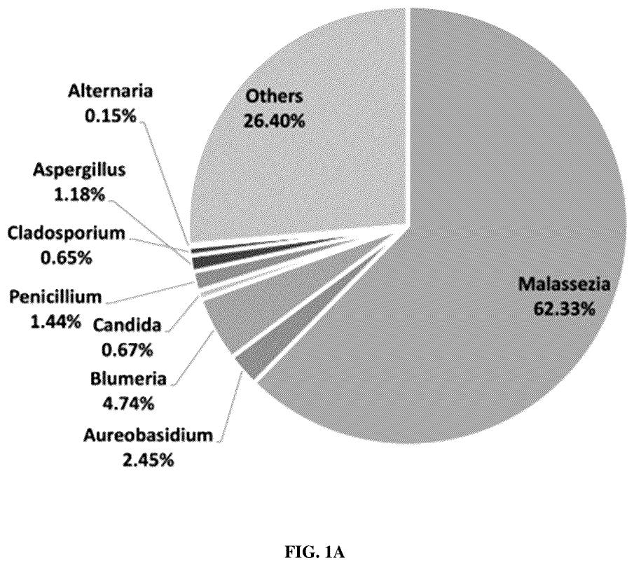

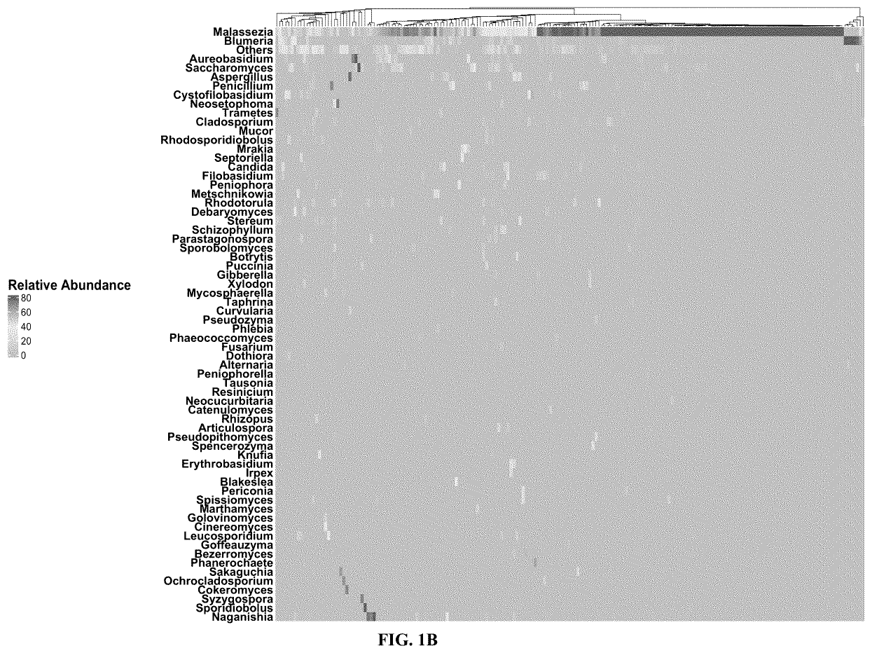

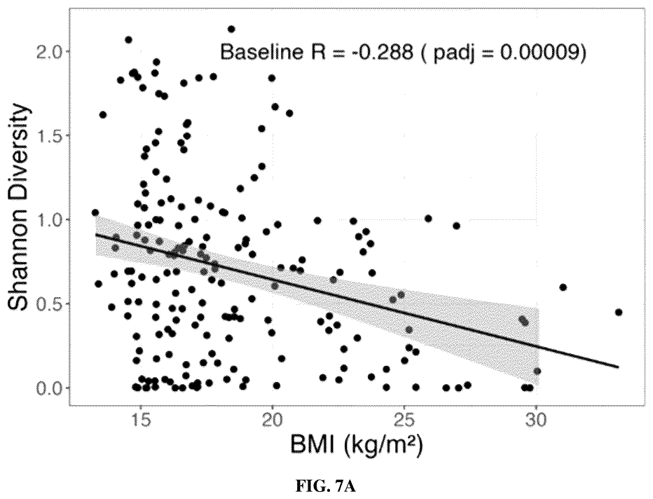

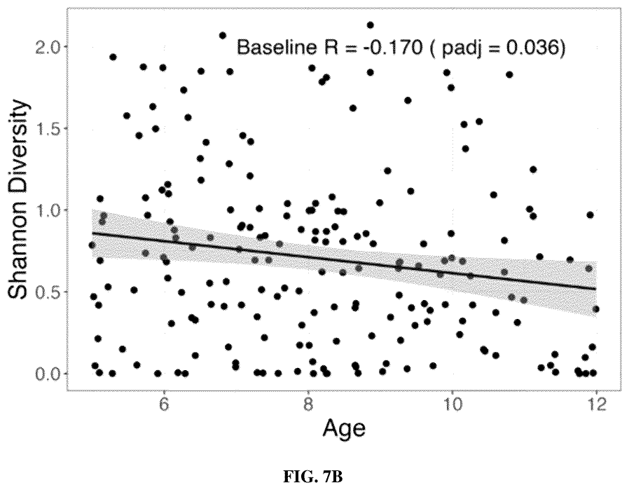

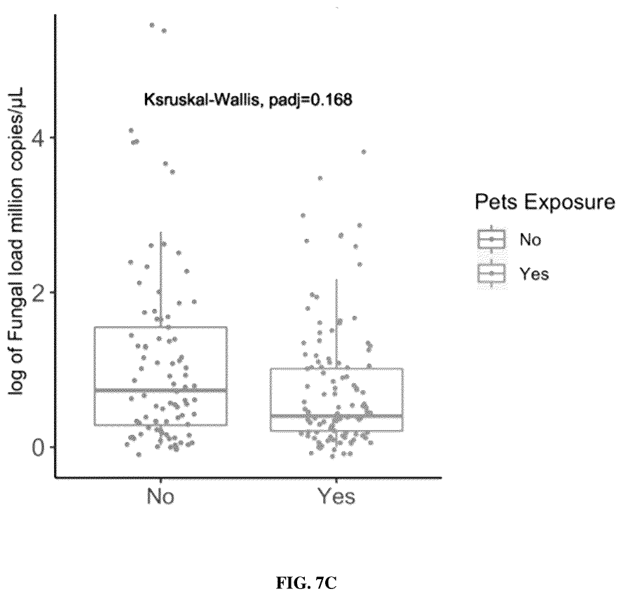

The upper airway mycobiota composition at baseline was first characterized. 499 fungal genera and 4704 ASVs from 194 baseline samples were identified. The lipophilic skin-commensal Malassezia was the most dominant fungal genus in the samples (62.33% of relative abundance) followed by Blumeria (4.74%), Aureobasidium (2.45%), Penicillium (1.44%), Aspergillus (1.18%), Candida (0.67%), Cladosporium (0.65%), and Alternaria (0.15%) (FIG. 1A). Unsupervised hierarchical clustering analysis revealed that the majority of children (154/194-79.4%) harbored a single, highly abundant genus Malassezia (FIG. 1B). The dominance by a single fungal genus in the mycobiota in the upper airway is different from the bacterial community of the same cohort in which several bacterial genera were dominant. Correlation analysis between Malassezia and baseline clinical factors showed that BMI and age were inversely correlated with fungal diversity while pet exposure was associated with lower fungal load (FIGS. 7A-7C).

### Associations Between Malassezia Species and Clinical Factors at Baseline

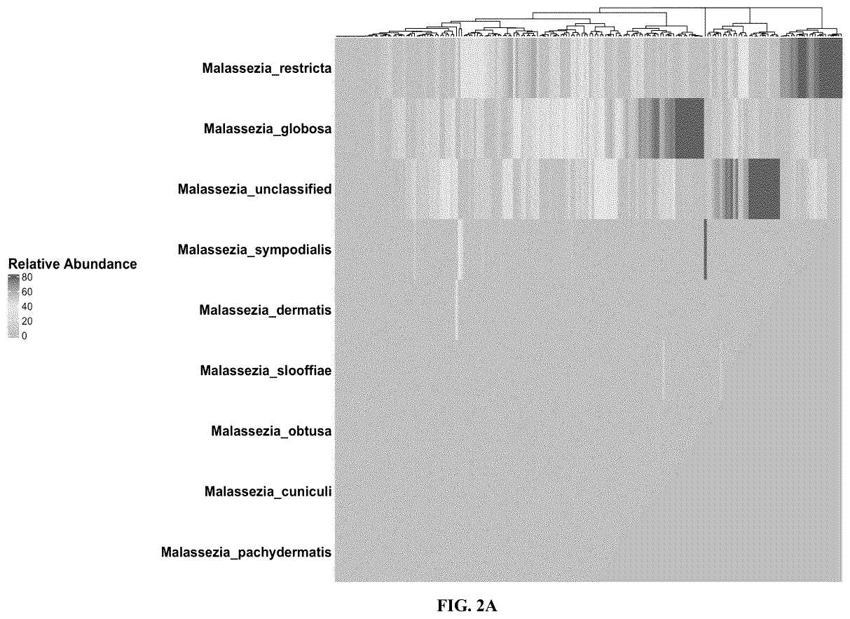

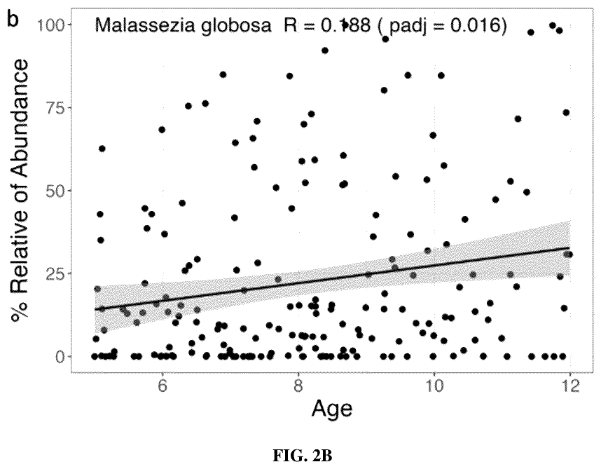

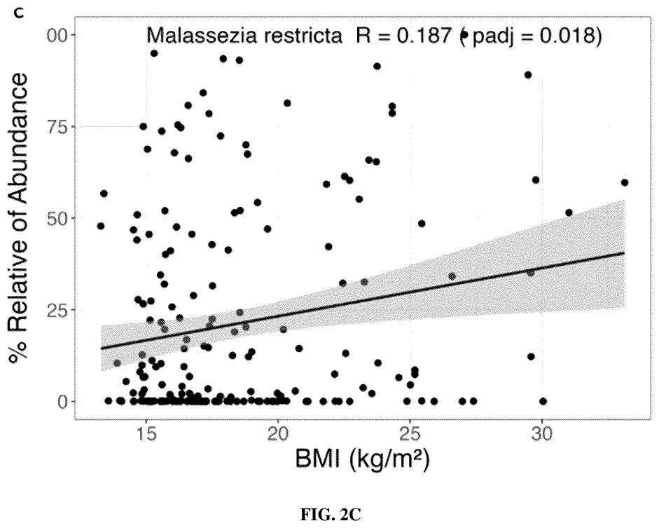

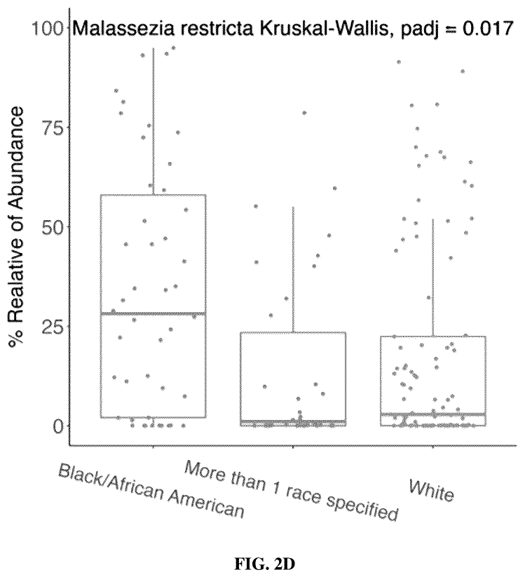

Since Malassezia was the most dominant genus at baseline, Malassezia species distribution patterns and their association with clinical variables were further examined. Eight different Malassezia species within the Malassezia genus were identified, with Malassezia globosa and Malassezia restricta being the most abundant (FIG. 2A). Malassezia globosa and Malassezia restricta had less than 90% identity in their ITS1 sequences, indicating a confident distinction between the two species by ITS1 sequencing. Malassezia globosa was positively correlated with age (padj=0.016, rho_{globosa}=0.1882, FIG. 2B). Alternatively, Malassezia restricta was positively correlated with BMI (padj=0.018, rho_{restricta}=0.187, FIG. 2C). Additionally, relative abundance of Malassezia restricta differed by race, as demonstrated by higher relative abundance in Black or African American children (FIG. 2D, padj=0.017), compared to White children and children that identified more than one race. Association between Malassezia species and other clinical variables was not shown. These findings suggest that depending on specific species, relative abundance of Malassezia species vary by age, BMI, and race.

Malassezia globosa is Associated with Better Asthma Control

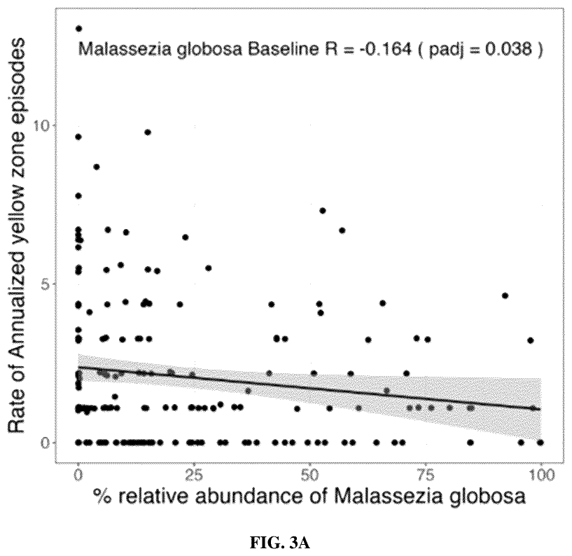

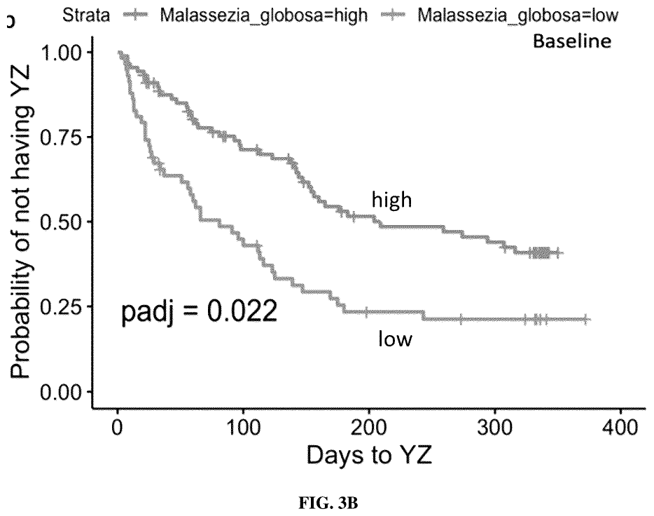

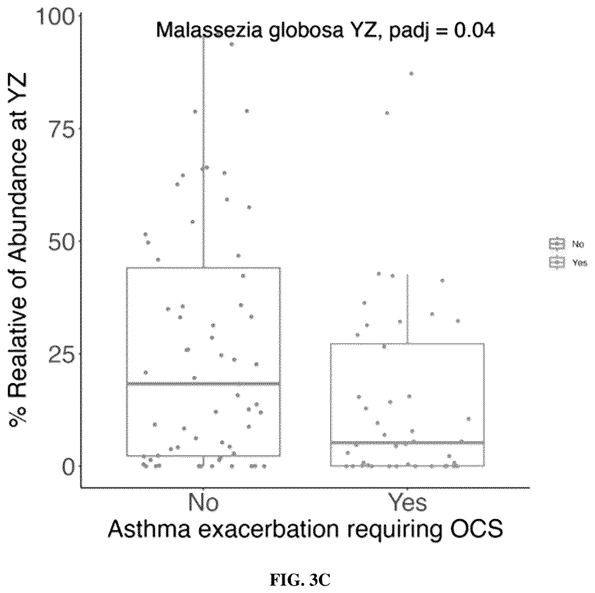

Among all children in the analysis, 69.6% experienced at least one episode of loss of asthma control (YZ). Linear regression analysis showed relative abundance of Malassezia globosa at baseline was negatively correlated with annualized episodes of YZ after controlling for age, pet exposure, BMI, or gender (p=0.038, FIG. 3A). These covariates are associated with the development of YZ in the cohort.

Although it previously reported that the presence or absence of respiratory virus in nasal blow samples at baseline was not associated with future loss of asthma control or exacerbation of asthma, it was still investigated if the fungal findings were related to the presence of respiratory virus, given important role of viruses in asthma development. After adjusting for the presence of respiratory virus, Malassezia globosa was still negatively associated with the annualized rate of YZ (p=0.0122). Children with higher relative abundance of Malassezia globosa at baseline also had longer time to develop their first episode of YZ compared to patients with lower relative abundance of Malassezia globosa (FIG. 3B, p=0.022), as shown by survival analysis with Cox proportional-hazards model.

To determine whether the Malassezia species were associated with severe asthma exacerbation when OCS therapy is required, relative abundance of Malassezia species at baseline or in YZ in children with and without asthma exacerbation was compared. 26.1% (26/214=26.1%) of participants had asthma exacerbation. It was found that the relative abundance of Malassezia globosa in the YZ was significantly lower in the children requiring OCS treatment after controlling for age, pets, BMI, virus presence, and gender in a linear regression analysis (FIG. 3C).

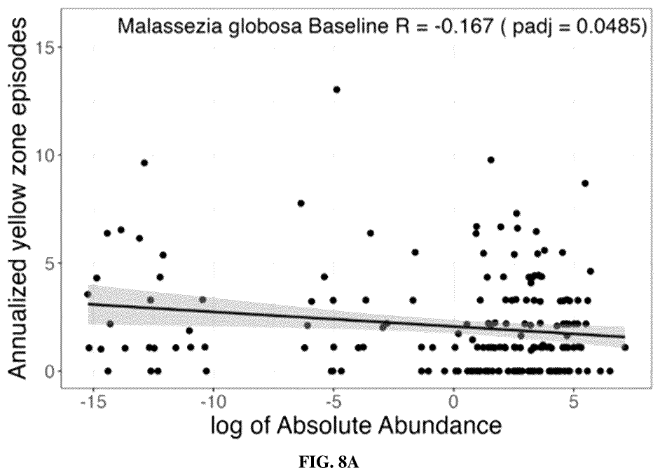

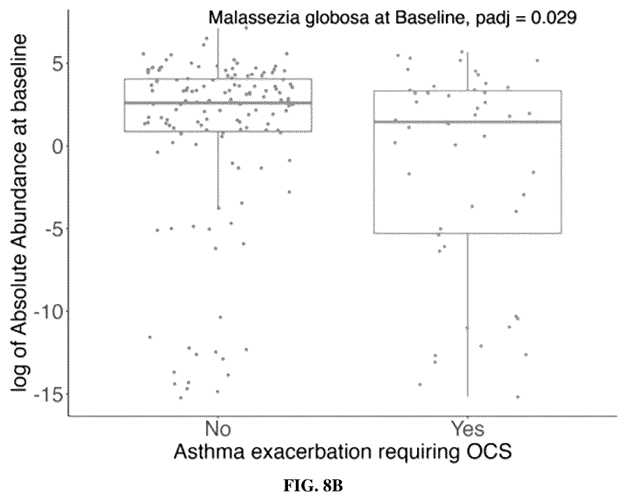

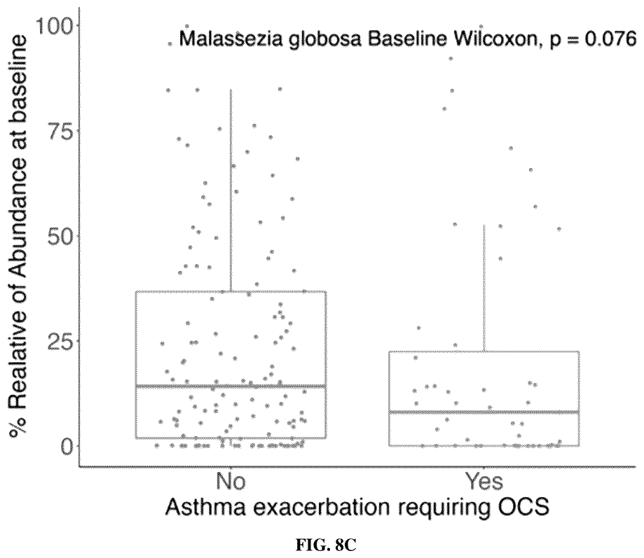

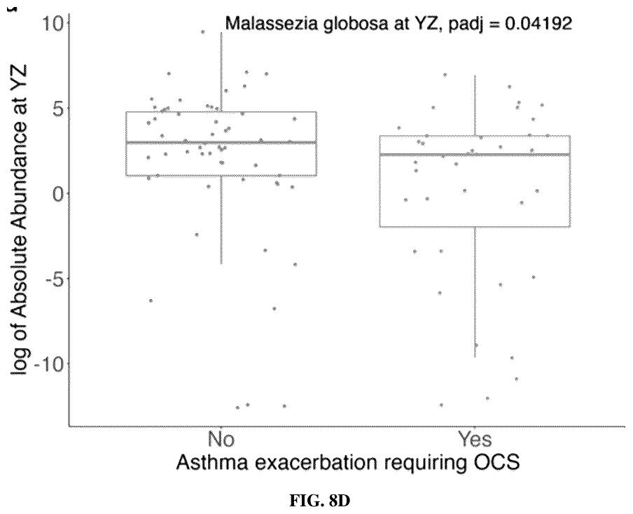

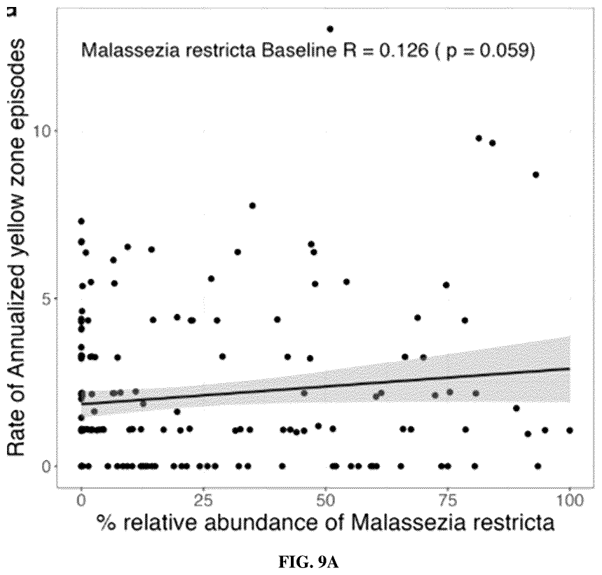

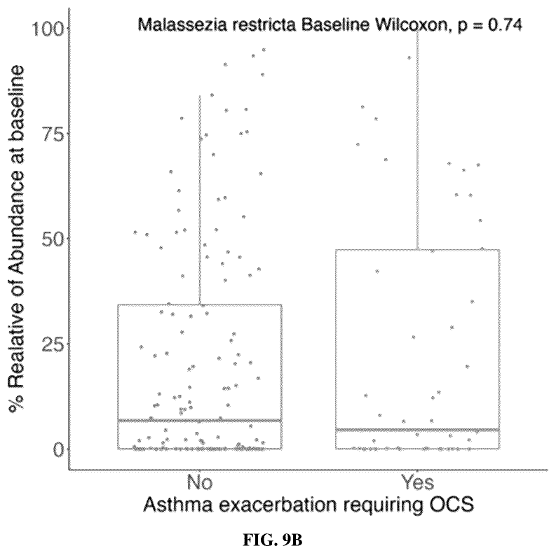

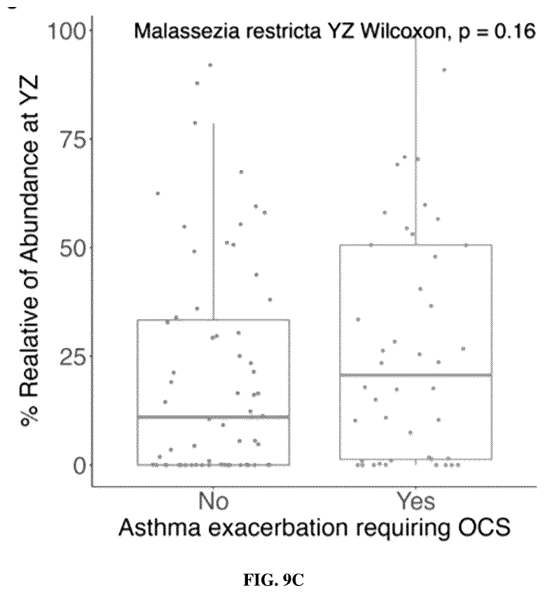

The absolute abundance of Malassezia species was further computed by multiplying their relative abundance with total fungal load. Results were consistent between the absolute abundance analysis and the relative abundance analysis (FIG. 8A). Additionally, the absolute abundance of Malassezia globosa at baseline was significant lower in children with and without asthma exacerbation (p=0.029, FIG. 8B), whereas relative abundance Malassezia globosa only showed marginal statistical difference between the two groups (p=0.076, FIG. 8C). However, no significant association between Malassezia restricta and loss of asthma control or future exacerbation of asthma was found (FIGS. 9A-9C).

As a sensitivity analysis, all samples from one geographic location were removed (9 locations in total) and the remaining samples were used to perform the above analyses (Table 2). The sensitivity tests by geographic sites showed largely consistent results for Malassezia globosa (Table 2).

TABLE 2
Sensitivity test of association of relative abundance of Malassezia globosa
at baseline/YZ and subsequent development of YZ or future asthma exacerbation*

| | All | Without | Without | Without | Without | Without | Without | Without | Without | Without |
|---|---|---|---|---|---|---|---|---|---|---|
| M. globosa | samples | SF | BOS | CHI | DEN | MSN | PIT | STL | TUS/RDU | INT/CHO |
| Samples no. (Baseline) | 194 | 179 | 180 | 182 | 169 | 177 | 177 | 162 | 173 | 153 |
| YZ episodes~M. globosa + | 0.038 | 0.029 | 0.019 | 0.048 | 0.049 | 0.064 | 0.038 | 0.046 | 0.002 | 0.210 |
| age + BMI + gender + pet | | | | | | | | | | |
| exposure + virus (P values) | | | | | | | | | | |
| YZ episodes~M. globosa + | −0.16 | −0.17 | −0.15 | −0.16 | −0.14 | −0.18 | −0.17 | −0.17 | −0.17 | −0.15 |
| age + BMI + gender + pet | | | | | | | | | | |
| exposure + virus | | | | | | | | | | |
| (Regression coefficient) | | | | | | | | | | |
| Samples no. (YZ) | 107 | 99 | 102 | 100 | 85 | 93 | 99 | 85 | 98 | 95 |
| OCS~M. globosa + age + | 0.040 | 0.038 | 0.028 | 0.024 | 0.053 | 0.046 | 0.149 | 0.029 | 0.131 | 0.049 |
| BMI + gender + pet | | | | | | | | | | |
| exposure + virus (P values) | | | | | | | | | | |
| OCS~M. globosa + age + | −1.91 | −2.16 | −2.09 | −2.18 | −1.96 | −2.15 | −1.34 | −2.45 | −1.4 | −1.85 |
| BMI + gender + pet | | | | | | | | | | |
| exposure + virus | | | | | | | | | | |
| (Regression coefficient) | | | | | | | | | | |

*Geographic locations abbreviation: SF (San Francisco), BOS (Boston), CHI (Chicago), DEN (Denver), MSN (Madison), PIT (Pittsburgh), STL (St. Louis), TUS/RDU (Tucson, Durham), INT/CHO (Winston-Salem, Charlottesville).

Taken together, the findings above suggest that Malassezia globosa in children is associated with a subsequently lower rate of YZ and lower risk of asthma exacerbation.

Dynamic Changes of Upper Airway Mycobiota from Baseline to YZ

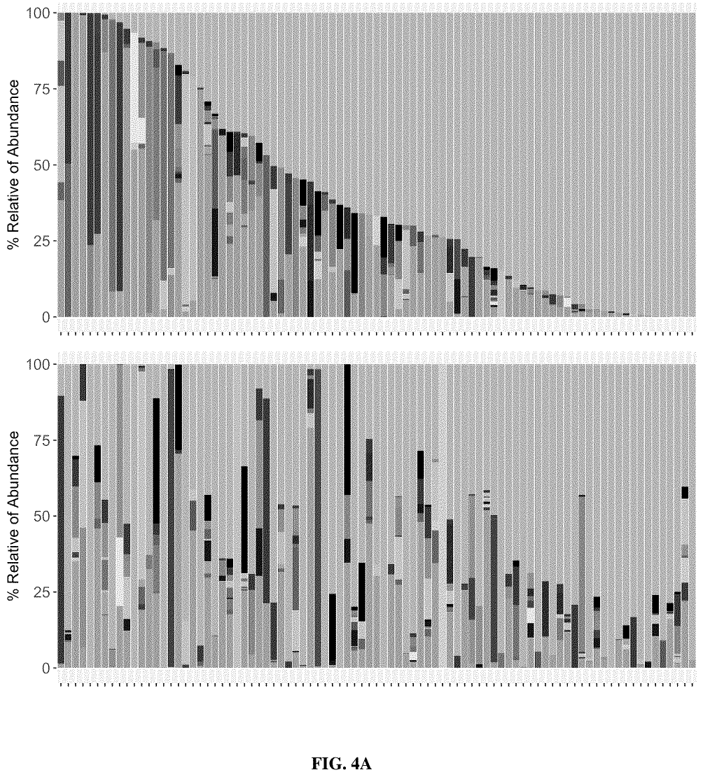

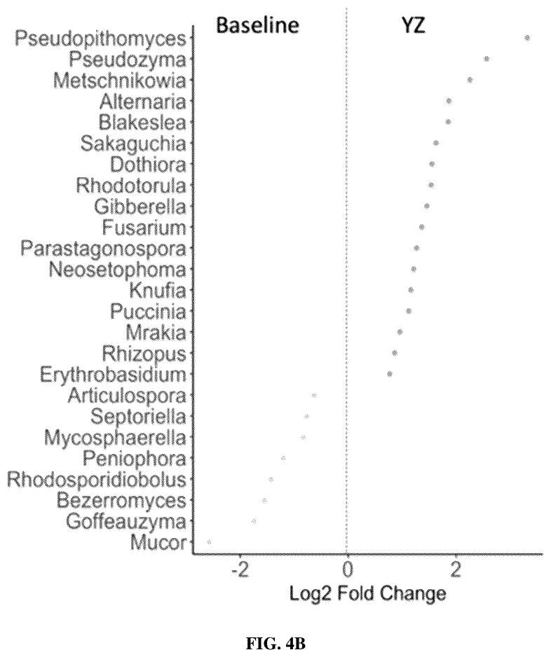

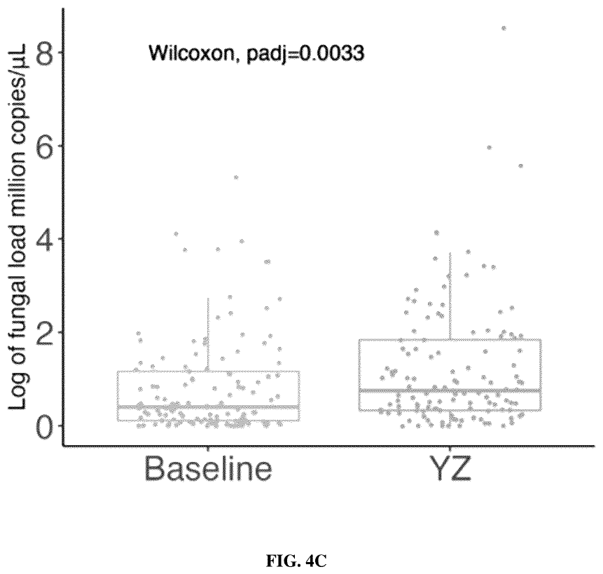

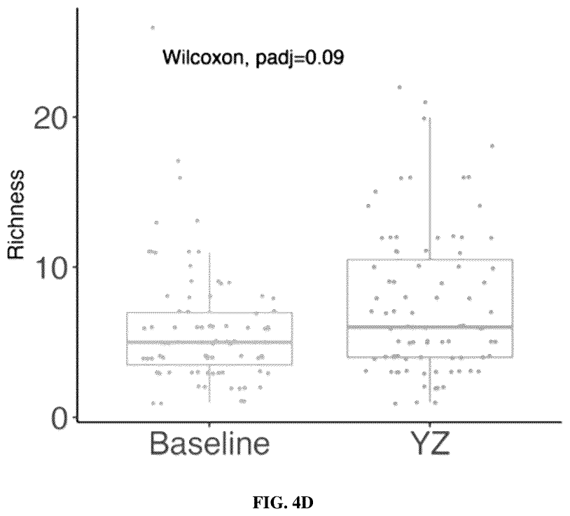

Leveraging the longitudinal design of the study, the changes of upper airway mycobiota from the baseline to YZ were determined in the 87 children who had samples at both timepoints. Significant changes in these genera from baseline to YZ were identified (FIG. 4A). Differential taxa analysis using DESeq2 analysis identified 20 significantly increased and 10 significantly decreased fungal genera at YZ (FIG. 4B; padj<0.05). Pseudopithomyces, which is linked to facial eczema in animals, showed the highest log 2 fold increase (3.32 folds) from baseline to YZ, followed by an environmental yeast Pseudozyma. Several previously reported asthma-associated fungi Alternaria and Fusarium also increased from baseline to YZ (FIG. 4B; padj<0.05). Alternatively, Mycosphaerella was significantly decreased at YZ in our cohort (FIG. 4B; padj<0.05). Mycosphaerella was reported to be negatively correlated with ICS use and enriched in patients with mild asthma or healthy control patients. Surprisingly, a mold Mucor was found significantly decreased at YZ.

Previous study showed that bacterial diversity and load were significantly increased from baseline to YZ. Total fungal load and fungal richness was significantly higher at YZ compared to baseline (FIGS. 4C-4D, padj=0.003 and 0.09, respectively, Wilcoxon rank-sum test). These results suggest an expansion of fungal microbiota from baseline to YZ.

### Relationship Between Fungi, Bacteria, and Viruses in the Upper Airway

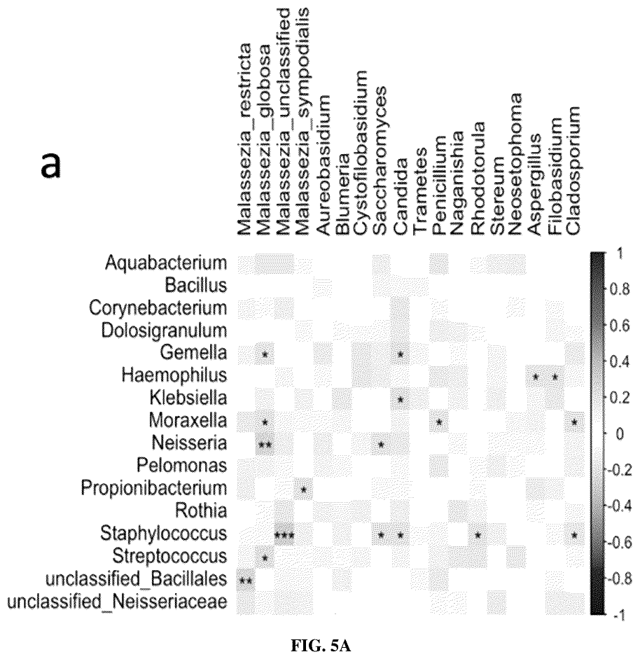

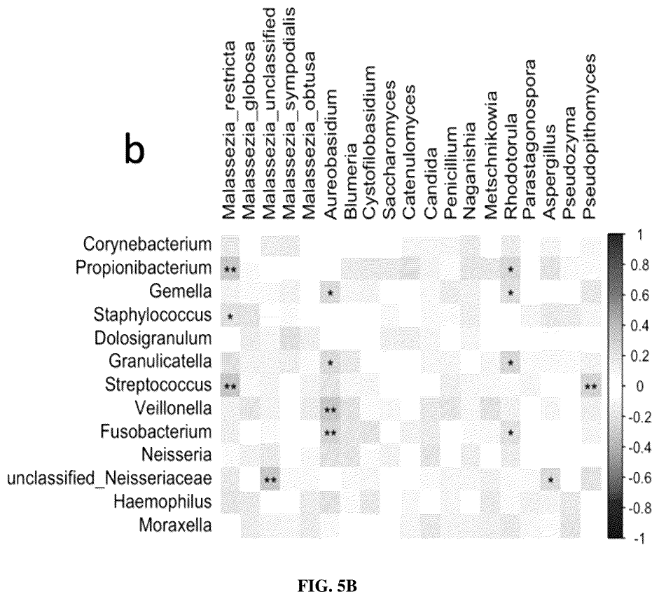

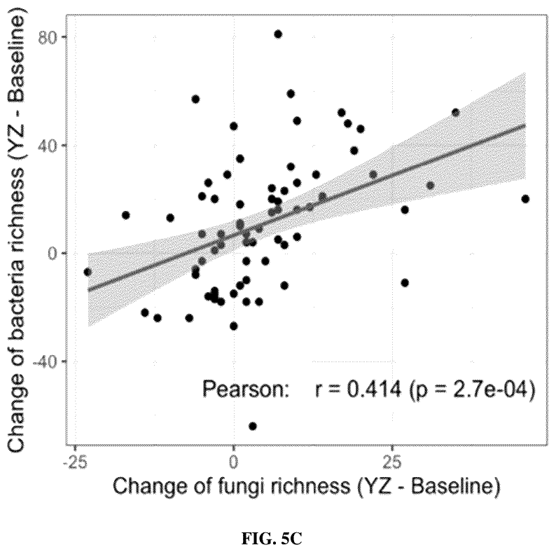

Paired-wise correlations, identified 288 and 247 bacteria-fungi correlations at baseline and YZ, respectively. At baseline, Malassezia globosa was negatively correlated with Moraxella (FIG. 5A), which is a previously reported pathogenic bacteria that is associated with loss of asthma control. Other interactions include positive correlations between Candida and common bacteria pathogens Klebsiella, Staphylococcus, and Gemella, whereas Aspergillus had a negative correlation with pathogenic Haemophilus.

At YZ, predominantly positive correlations between potentially pathogenic fungi and bacteria (60.3% correlations) were identified. For example, fungus Aureobasidium had strong positive correlations with Fusobacterium and Veillonella. Fungal genus Pseudopithomyces, that also increased in YZ (FIG. 5B), had strong positive correlations with Streptococcus that was also increased in YZ. Correlations between fungi and bacteria at YZ likely reflect concomitant increase of fungi and bacteria in response to YZ. Indeed, increased fungal diversity was positively correlated with increased bacterial diversity from baseline to YZ (FIG. 5C, p=2.7e-04, rho-0.414). These results suggest a complex co-occurrent and co-exclusive relationships between fungi and bacteria and distinct correlation patterns between baseline and YZ in the upper airway.

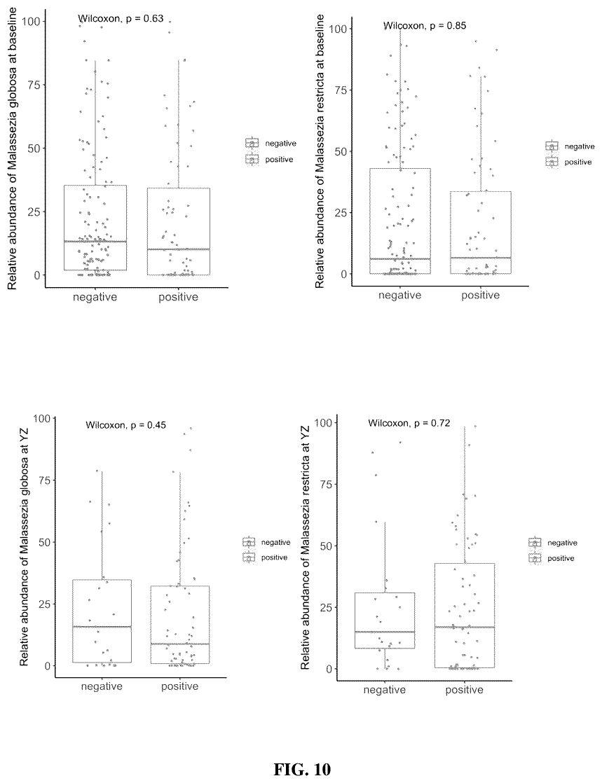

Fungal diversity and specific fungal compositions were not statistically different between virus-positive and -negative samples at either baseline or YZ (FIG. 10).

### Discussion

The present study defined the mycobiota characteristics, dynamics, and trans-kingdom interactions in the upper airway of school-aged children with asthma, and provided the first novel evidence for associations between the airway fungi and asthma control. The present inventors found an association between abundance of Malassezia globosa and lower rate of annualized episodes of YZ, along with a lower risk of progression from YZ to severe asthma exacerbation. The findings represent the role of the commensal fungal mycobiome in asthma.

Malassezia is a lipid-dependent basidiomycetous yeast and is one of the most common fungi residing on human skin. The present inventors found Malassezia to be the most abundant fungal genera in nasal blow samples, which is consistent with the other studies. The anterior nares of the nasal passage are lined with keratinized squamous epithelium. Lipid-rich mucosa of the anterior nares can promote growth of lipophilic fungi such as Malassezia. Association between age with Malassezia is also evident from a study with a cohort of children from 6-17 years old, which is likely due to increasing sebum production and sex hormone concentrations from early childhood to puberty. The positive correlation between Malassezia and BMI may also be related to higher sebum production in participants with higher BMI. Nevertheless, after controlling for age and BMI, association between the abundance of Malassezia species in YZ and asthma exacerbation remained significant, suggesting that Malassezia species may be related to asthma symptoms and exacerbation independent of age and BMI.

Malassezia is considered a commensal member of the human microbiome which has been significantly understudied. Its functional significance is largely derived from studies in skin diseases. For the first time, the present inventors reported that increased relative abundance of M. globosa in the upper airway is associated with better control of asthma in asthmatic children. One possible mechanism is through actively preventing or inhibiting pathogen colonization. For example, on skin, Malassezia can prevent colonization by pathogenic Staphylococcus aureus by secreting proteases. Interestingly, the correlation network analysis identified a co-exclusion relationship between M. globosa and bacterial pathogen Morexella that may be related to asthma exacerbation, supporting a competitive role of specific fungi and bacteria in asthma. Alternatively, M. globosa can indirectly suppress inflammation, such as by inducing anti-inflammatory cytokine IL-10 observed in keratinocytes following exposure to M. globosa.

The present study also identified cross-kingdom interactions between bacteria and fungi, but not between viruses and fungi, in the airways of children with asthma. Co-occurrence and co-exclusive relationship between fungi and bacteria suggest these organisms may work cooperatively or competitively in driving asthma pathogenesis. However, these relationships may be a result of nutritional preference of these micro-organisms. For example, co-occurrence of the organisms may simply reflect similar characteristics in nutrient utilization. Lack of correlations between viruses and fungi at baseline may also be due to no active viral infection at a time when asthma symptoms were well-controlled. Over 70% of children were virus-positive at YZ, resulting in less statistical power in detecting any fungi difference in viral-positive and -negative groups. Subsequent in vitro and in vivo work are warranted to study cross-kingdom interactions and host immune-microbiome interactions, allowing for mechanistic understanding of how fungi influence pathogenesis of asthma.

The patient population of this study included asthmatic children who were all treated with low doses of ICS, which may have an effect on the upper airway fungal profile. However, this is unlikely to affect the main findings of the study—the associations between Malassezia globosa abundance and loss of asthma control—as all study participants received this daily low dose ICS therapy. The study did not include lower airway samples as the upper airways are increasingly recognized as an important gatekeeper to respiratory health. The upper airway samples are easy to access, rendering in wider clinical application than lower airway samples.

Overall, there is a considerable knowledge gap of the commensal fungal microbiome in the upper airway in asthmatic children. This disclosure establishes associations between airway mycobiota and loss of asthma control in asthmatic children. These associations may allow for future mechanistic studies to better understand the role of commensal fungi in the pathogenesis of asthma, and open novel avenues to develop mycobiota-associated biomarkers to predict and monitor disease outcomes.

Compounds and materials are described using standard nomenclature. Unless defined otherwise, all technical and scientific terms used herein have the same meaning as is commonly understood by one of skill in the art to which this invention belongs.

The following terms are used to describe the invention of the present disclosure. In instances where a term is not specifically defined herein, that term is given an art-recognized meaning by those of ordinary skill applying that term in context to its use in describing the present disclosure.

The use of the terms “a” and “an” and “the” and similar referents (especially in the context of the following claims) are to be construed to cover both the singular and the plural, unless otherwise indicated herein or clearly contradicted by context. By way of example, “an element” means one element or more than one element.

It should also be understood that, in certain methods described herein that include more than one step or act, the order of the steps or acts of the method is not necessarily limited to the order in which the steps or acts of the method are recited unless the context indicates otherwise. Furthermore, the terms first, second, etc., as used herein are not meant to denote any particular ordering, but simply for convenience to denote a plurality of, for example, layers.

The terms “comprising”, “having”, “including”, and “containing” are to be construed as open-ended terms (i.e., meaning “including, but not limited to”) unless otherwise noted.

The terms “about” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within +10% or 5% of the stated value. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “one or more,” as used herein, means at least one, and thus includes individual components as well as mixtures/combinations of the listed components in any combination.

Other than in the operating examples, or where otherwise indicated, all numbers expressing quantities of ingredients and/or reaction conditions are to be understood as being modified in all instances by the term “about,” meaning within 10% of the indicated number (e.g., “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

As used herein, the term “administering” means the actual physical introduction of a composition into or onto (as appropriate) a subject, a host or cell. Any and all methods of introducing the composition into the subject, host or cell are contemplated according to the invention; the method is not dependent on any particular means of introduction and is not to be so construed. Means of introduction are well-known to those skilled in the art, and also are exemplified herein.

As used herein, “optional” or “optionally” means that the subsequently described event or circumstance may or may not occur, and that the description includes instances where said event or circumstance occurs and instances where it does not.

As used herein, the term “pharmaceutically acceptable” refers to compositions that are physiologically tolerable and do not typically produce an allergic or similar untoward reaction when administered to a subject, preferably a human subject. Preferably, as used herein, the term “pharmaceutically acceptable” means approved by a regulatory agency of a federal or state government or listed in the U.S. Pharmacopeia or other generally recognized pharmacopeia for use in animals, and more particularly in humans.

As used herein, the terms “treat,” “treating,” and “treatment” include inhibiting the pathological condition, disorder, or disease, e.g., arresting or reducing the development of the pathological condition, disorder, or disease or its clinical symptoms; or relieving the pathological condition, disorder, or disease, e.g., causing regression of the pathological condition, disorder, or disease or its clinical symptoms. These terms also encompass therapy and cure. Treatment means any way the symptoms of a pathological condition, disorder, or disease are ameliorated or otherwise beneficially altered. Preferably, the subject in need of such treatment is a mammal, preferably a human.

As used herein, “preventing” or “prevention” refers to a reduction in risk of acquiring a disease or disorder (i.e., causing at least one of the clinical symptoms of the disease not to develop in a patient that may be exposed to or predisposed to the disease but does not yet experience or display symptoms of the disease). Prevention does not require that the disease or condition never occurs in the subject. Prevention includes delaying the onset or severity of the disease or condition.

As used herein, a “predetermined threshold value” or “threshold value” of a fungal species refers to the level of the fungal species in a corresponding control sample or group of control samples obtained from, for example, a normal, healthy subject not afflicted with a disease associated with inflammation (e.g., asthma), a subject having never been diagnosed with a disease associated with inflammation (e.g., asthma), a subject previously diagnosed with a disease associated with inflammation (e.g., asthma) whose disease state has not progressed, a subject previously diagnosed with a disease associated with inflammation (e.g., asthma) whose disease state has progressed (e.g., with treatment, or with or without receiving treatment), or a subject from an earlier time point, e.g., prior to treatment, an earlier assessment time point, at an earlier stage of treatment, or prior to onset of the disease. The predetermined threshold value may be determined prior to or concurrently with measurement of fungi species levels in a biological sample. The control sample may be from the same subject at a previous time or from different subjects.

As used herein, the term “effective amount” refers to the amount of a therapy, which is sufficient to reduce or ameliorate the severity and/or duration of a disorder or one or more symptoms thereof, inhibit or prevent the advancement of a disorder, cause regression of a disorder, inhibit or prevent the recurrence, development, onset or progression of one or more symptoms associated with a disorder, detect a disorder, or enhance or improve the prophylactic or therapeutic effect(s) of another therapy (e.g., prophylactic or therapeutic agent). An effective amount can require more than one dose.

Effective amounts may vary depending upon the biological effect desired in the individual, condition to be treated, and/or the specific characteristics of the composition according to the present invention and the individual. In this respect, any suitable dose of the composition can be administered to the patient (e.g., human), according to the type of disease to be treated. Various general considerations taken into account in determining the “effective amount” are known to those of skill in the art and are described, e.g., in Gilman et al., eds., Goodman And Gilman's: The Pharmacological Bases of Therapeutics, 8th ed., Pergamon Press, 1990; and Remington's Pharmaceutical Sciences, 17th Ed., Mack Publishing Co., Easton, Pa., 1990, each of which is herein incorporated by reference. The dose of the composition according to the present invention desirably comprises about 0.1 mg per kilogram (kg) of the body weight of the patient (mg/kg) to about 400 mg/kg (e.g., about 0.75 mg/kg, about 5 mg/kg, about 30 mg/kg, about 75 mg/kg, about 100 mg/kg, about 200 mg/kg, or about 300 mg/kg). In another embodiment, the dose of the composition according to the present invention comprises about 0.5 mg/kg to about 300 mg/kg (e.g., about 0.75 mg/kg, about 5 mg/kg, about 50 mg/kg, about 100 mg/kg, or about 200 mg/kg), about 10 mg/kg to about 200 mg/kg (e.g., about 25 mg/kg, about 75 mg/kg, or about 150 mg/kg), or about 50 mg/kg to about 100 mg/kg (e.g., about 60 mg/kg, about 70 mg/kg, or about 90 mg/kg).

The term “subject” is used herein to refer to an animal, such as a mammal, including a primate (such as a human, a non-human primate, e.g., a monkey, and a chimpanzee), a non-primate (such as a cow, a pig, a camel, a llama, a horse, a goat, a rabbit, a sheep, a hamster, a guinea pig, a cat, a dog, a rat, a mouse, and a whale), a bird (e.g., a duck or a goose), and a shark. In an embodiment, the subject is a human, such as a human being treated or assessed for a disease, disorder or condition, a human at risk for a disease, disorder or condition, a human having a disease, disorder or condition, and/or human being treated for a disease, disorder or condition as described herein. In some embodiments, the subject does not suffer from an ongoing autoimmune disease. In one embodiment, the subject is about 1, 2, 3, 4, 5, 6, 7, 8, 9, or 10 years of age. In another embodiment, the subject is about 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100 years of age. Values and ranges intermediate to the above recited ranges are also intended to be part of this invention. In addition, ranges of values using a combination of any of the above-recited values as upper and/or lower limits are intended to be included.

As used herein, a “symptom” of a disease includes any clinical or laboratory manifestation associated with the disease, and is not limited to what a subject can feel or observe.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art of this disclosure.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

### INCORPORATION BY REFERENCE

All U.S. and PCT patent publications and U.S. patents mentioned herein are hereby incorporated by reference in their entirety as if each individual patent publication or patent was specifically and individually indicated to be incorporated by reference. In case of conflict, the present application, including any definitions herein, will control.

### Other Embodiments

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

### REFERENCE

1. Zhou Y, et al. The upper-airway microbiota and loss of asthma control among asthmatic children. Nat Commun. 2019; 10 (1): 5714. doi: 10.1038/s41467-019-13698-x
2. Jackson D J. et al. Quintupling Inhaled Glucocorticoids to Prevent Childhood Asthma Exacerbations. N Engl J Med. 2018: 378 (10): 891-901. doi: 10.1056/NEJMoa1710988

1. A method for prognosing the risk for progression of a disease or disorder associated with inflammation in a subject, comprising:
(a) detecting the level of a fungal species in a biological sample from the subject, wherein the fungal species comprises Malassezia globosa, and
(b) comparing the level of the fungal species in the biological sample with a predetermined threshold value;
wherein the level of the fungal species above or below the predetermined threshold value indicates that the subject is at risk for progression of the disease or disorder associated with inflammation.

2. The method of claim 1, wherein the disease or disorder associated with inflammation is selected from the group consisting of asthma, inflammatory bowel disease, rheumatoid arthritis, juvenile rheumatoid arthritis, psoriatic arthritis, osteoarthritis, refractory rheumatoid arthritis, chronic non-rheumatoid arthritis, osteoporosis or bone resorption, coronary heart disease, atherosclerosis, endothelial dysfunction, vasculitis, ulcerative colitis, psoriasis, adult respiratory distress syndrome, diabetes, delayed-type hypersensitivity in skin disorders, endothelial dysfunction, or a combination thereof.

3. The method of claim 1, wherein the disease or disorder associated with inflammation is asthma.

4. The method of claim 1, wherein:
a decrease in the level of Malassezia globosa is indicative of progression of the disease or disorder associated with inflammation in the subject;
the level of Malassezia globosa is detected by quantitative PCR and/or internal transcribed spacer (ITS) sequencing; and/or
the level of Malassezia globosa is detected by ITS1 sequencing.

5. (canceled)

6. (canceled)

7. The method of claim 1, wherein the biological sample comprises a nasal saline wash, a nasal blow sample, a nasal mucus, a nasal discharge, a sinus mucus, or a sinus brushing comprising mucus from the surface of a sinus, or a bodily fluid.

8. The method of claim 1, further comprising:
selecting a treatment regimen based on the risk of progression of the disease or disorder associated with inflammation in the subject, and/or
obtaining a biological sample from the subject.

9. The method of claim 8, wherein the treatment regimen is selected from the group consisting of oral corticosteroids (OCS), inhaled corticosteroids, leukotriene modifiers, anticholinergic agents, short-acting beta agonists, long-acting beta agonists, short-acting muscarinic antagonists, long-acting muscarinic antagonists, Theophylline, or any combination thereof.

10. (canceled)

11. The method of claim 1, wherein the subject is a human subject.

12. The method of claim 1, wherein:
the subject has been previously diagnosed with the disease or disorder associated with inflammation;
the subject is concurrently diagnosed with the disease or disorder associated with inflammation;
the subject has been previously treated for the disease or disorder associated with inflammation; and/or
the subject has not yet been treated for the disease or disorder associated with inflammation.

13-16. (canceled)

17. The method of claim 3, wherein:
the progression of asthma comprises an increased rate of annualized episodes of yellow zone (YZ) or a loss of asthma control; and/or
the progression of asthma comprises an increased risk of progression from YZ to severe asthma exacerbation.

18-30. (canceled)

31. A method for monitoring a disease or disorder associated with inflammation in a subject, comprising,
(a) detecting the level of a fungal species in a first biological sample obtained at a first time from the subject having the disease or disorder associated with inflammation, wherein the fungal species comprises Malassezia globosa, and
(b) detecting the level of the fungal species in a second biological sample obtained from the subject at a second time, wherein the second time is later than the first time; and
(c) comparing the level of the fungal species in the second sample with the level of the fungal species in the first sample;
wherein a change in the level of the fungal species is indicative of progression of the disease or disorder associated with inflammation in the subject.

32. The method of claim 31, wherein the disease or disorder associated with inflammation is selected from the group consisting of asthma, inflammatory bowel disease, rheumatoid arthritis, juvenile rheumatoid arthritis, psoriatic arthritis, osteoarthritis, refractory rheumatoid arthritis, chronic non-rheumatoid arthritis, osteoporosis or bone resorption, coronary heart disease, atherosclerosis, endothelial dysfunction, vasculitis, ulcerative colitis, psoriasis, adult respiratory distress syndrome, diabetes, delayed-type hypersensitivity in skin disorders, endothelial dysfunction, or a combination thereof.

33. The method of claim 32, wherein the disease or disorder associated with inflammation is asthma.

34. The method of claim 31, wherein:
a decrease in the level of Malassezia globosa is indicative of progression of the disease or disorder associated with inflammation in the subject;
the level of Malassezia globosa is detected by quantitative PCR and/or internal transcribed spacer (ITS) sequencing; and/or
the level of Malassezia globosa is detected by ITS1 sequencing.

35. (canceled)

36. (canceled)

37. The method of claim 31, wherein the first and the second biological samples comprise a nasal saline wash, a nasal blow sample, a nasal mucus, a nasal discharge, a sinus mucus, or a sinus brushing comprising mucus from the surface of a sinus, or a bodily fluid.

38. (canceled)

39. A method for treating, preventing or slowing down the progression of a disease or disorder associated with inflammation in a subject, comprising increasing the level of a fungal species in the subject, and wherein the fungal species comprises Malassezia globosa.

40. The method of claim 39, wherein increasing the level of a fungal species in the subject comprises administering to the subject an agent that increases the level of the fungal species.

41. (canceled)

42. The method of claim 39, wherein the disease or disorder associated with inflammation is selected from the group consisting of asthma, inflammatory bowel disease, rheumatoid arthritis, juvenile rheumatoid arthritis, psoriatic arthritis, osteoarthritis, refractory rheumatoid arthritis, chronic non-rheumatoid arthritis, osteoporosis or bone resorption, coronary heart disease, atherosclerosis, endothelial dysfunction, vasculitis, ulcerative colitis, psoriasis, adult respiratory distress syndrome, diabetes, delayed-type hypersensitivity in skin disorders, endothelial dysfunction, or a combination thereof.

43. (canceled)

44. The method of claim 1,
wherein the method further comprises step (c) of selecting a treatment regimen based on the risk of progression of the disease or disorder associated with inflammation in the subject.

45. The method of claim 1,
wherein the disease or disorder associated with inflammation is asthma, and
wherein the method further comprises step (c) of selecting a treatment regimen based on the risk of progression of asthma in the subject.

46. (canceled)


SEQUENCE LISTING (2 sequences)

SEQ ID NO: 1 - DNA, 22 bp, synthetic construct
  source 1..22: mol_type = other DNA
gtaaaagtcgtaacaaggtttc

SEQ ID NO: 2 - DNA, 22 bp, synthetic construct
  source 1..22: mol_type = other DNA
gttcaaagaytcgatgattcac